(19) United States
(12) Patent Application Publication
(10) Pub. No.: US 2023/0260043 A1
(43) Pub. Date: Aug. 17, 2023

(54) COMPUTING SYSTEM IMPLEMENTING A COGNITIVE-BASED RISK ASSESSMENT SERVICE FOR MOTOR VEHICLE RISK DETERMINATION

(71) Applicant: Hi.Q, Inc., Plano, TX (US)

(72) Inventors: Munjal Shah, Plano, TX (US); Gaurav Suri, Plano, TX (US); Kurt Roots, Plano, TX (US); Ryan Hinchey, Plano, TX (US)

(21) Appl. No.: 18/012,200

(22) PCT Filed: Jun. 21, 2021
(86) PCT No.: PCT/US21/38287

§ 371 Date: Dec. 21, 2022

Related U.S. Application Data
(60) Provisional application No. 63/042,488, filed on Jun. 22, 2020.

(51) Int. Cl. G06Q 40/08; G16H 50/30

(52) U.S. Cl. CPC G06Q 40/08; G16H 50/30

(57) ABSTRACT

A computing system can implement a cognitive-based risk mitigation service. The system can train and test a correlation model using a cognitive assessment test (CAT) distributed to control base users. The control base users may further provide access to motor vehicle records such that the system can determine a set of correlations between cognitive health metrics tested by the (CAT) and vehicle accident risk as indicated by actual accident data in the motor vehicle records. Once a threshold level of accuracy is attained, the correlation model may be executed on response data from inquiring users taking the CAT to determine motor vehicle risk of those users without them providing access to their motor vehicle records. This motor vehicle risk may then be used to generate a risk mitigation policy package for the inquiring user.

### CROSS-REFERENCE TO RELATED APPLICATION

This application claims the benefit of priority to U.S. Provisional Application No. 63/042,488, filed on Jun. 22, 2020, which is hereby incorporated by reference in its entirety.

### BACKGROUND

Risk assessment techniques have typically involved generalizations with respect to factors such as health, age, income, assets, liabilities, etc. With the advent of big data and computer modeling techniques, risk assessment has become more granular, with improvements in such techniques providing more precision and efficiency for consumers of risk mitigation products.

### BRIEF DESCRIPTION OF THE DRAWINGS

The disclosure herein is illustrated by way of example, and not by way of limitation, in the figures of the accompanying drawings in which like reference numerals refer to similar elements, and in which:

FIG. 1 illustrates a network-based computing system executing a cognition correlation model to generate a cognitive assessment test for determining individualized vehicle accident risk for consumers, according to examples described herein;

FIG. 2 illustrates client computing device for accessing the cognitive assessment test and engaging in an insurance coverage service provided by the network-based computing system, according to examples described herein;

FIG. 3 is a flow chart describing an example method for generating cognitive assessment test through execution of a cognition correlation model and determining individual vehicle accident risk, according to example described herein; and

FIG. 4 is a block diagram that illustrates a computer system upon which examples described herein may be implemented.

### DETAILED DESCRIPTION

A network-based computing system can operate to process motor vehicle record (MVR) information of a control base of users (e.g., purportedly high-risk senior individuals over sixty-five years old) over a period of time (e.g., three previous years) to identify any recorded motor vehicle incidents for each control base user. In further implementations, the computing system can ingest additional data that may be indicative of motor vehicle incidents, such as on-board sensor information from their vehicles (e.g., video data that has been flagged as risky, such as interior video data showing lack of alertness and/or exterior video data indicating lane drift, close calls, braking incidences, running stop signs or red lights, hitting objects or curbs, and the like). This control base of users—which can comprise upwards of hundreds, thousands, tens of thousands, or more users and/or volunteers—may then be tasked to take a cognitive assessment test (CAT) comprised of cognitive assessment problems, questions, or assertions.

The CAT may be taken remotely and electronically through a website or via a user interface of a dedicated cognitive assessment application developed by a risk mitigation entity that updates and manages the computing system described herein. CAT responses can comprise each control base individual's response to each CAT question or assertion. The CAT can comprise filtered questions and assertions (e.g., a multiple-choice quiz) that test various cognitive health metrics of the users, such as spatial reasoning, motor function, word-finding and language processing ability, and time to completion for each question/assertion as well as the overcall CAT. As such, CAT performance of the individuals in the control base can enable the computing system to generally measure the cognitive well-being of the control base individuals (e.g., comprised of senior citizens) by digitally assessing memory and recall, word finding abilities, reaction time and motor function, and the like.

According to examples described herein, the computing system can execute one or more correlation models that compare(s) the CAT results of the control base users with the MVR information and/or the additional motor vehicle data (e.g., historical on-board sensor data) to determine a set of cognitive correlations between the cognitive health metrics of the individuals tested by the CAT and the historical accident information provided by the MVR information of those control base individuals. Execution of the correlation model(s) can include a training and testing phase in which the data set is randomly partitioned on a 90/10-train/test manner and iteratively executed on multiple occasions (e.g., 100 times) to establish a median model accuracy utilizing an area under curve (AUC) score to determine overall model accuracy. In various implementations, several correlation models can be evaluated using the same data set. In certain aspects, the data set can comprise an imbalanced data set where accidents and/or vehicle incidents in general represent a minority class of the total (e.g., less than 10%).

It is contemplated that this approach can represent a problem for the decision function of common machine learning models like decision trees. In an imbalanced data set, the use of such an imbalanced data set as a classifier will tend to favor the majority classes. To solve this problem, the computing system implements a Balanced Random Forest Classifier, which comprises an ensemble method where each tree of the forest (or decision tree) is provided a balanced bootstrap sample. In various examples, the computing system can generate a confusion matrix for each trained and tested model to provide the respective proportions of true negatives, false positives, false negatives, and true positives.

Accordingly, the computing system can determine an accuracy for each trained and tested cognitive correlation model, the most accurate of which can be utilized by the computing system for assessing new, inquiring users that have not participated in the control base testing phase. Thus, the inquiring users can take the CAT and provide responses, which can be analyzed through execution of the accurate cognitive correlation model to generate a set of cognitive risk scores for the inquiring user. As provided herein, this set of cognitive risk scores can correlate directly with vehicular accident risk, and may be used by the computing system to generate a risk mitigation policy or underwriting class for the user. In certain examples, the risk mitigation policy can be individualized for the user or can be a tiered class of risk mitigation policies for a like cluster of users (e.g., an underwriting class) having similar cognitive risk scores.

As described herein, subsequent to the training and testing phase, it has been observed that the primary factors within the CAT that correlate most significantly to vehicular accidents relate to questions involving spatial reasoning, motor function, word-finding and language comprehension, and time to completion of the CAT. Specifically, certain cognitive feature descriptions have p-values of less than 0.05. One such cognitive description is spatial reasoning ability, such as total amount of movement for a step in a spatial task (e.g., the capacity to understand, reason, and remember the spatial relations among objects or space), the amount of time to complete each step for a particular spatial task, the total amount of time to complete the entire spatial task, and the mean amount of time to complete a step within a spatial task.

Additional feature descriptions having p-values of less than 0.05 include executive function (e.g., a set of cognitive processes that are necessary for the cognitive control of behavior). These executive functions include basic cognitive processes such as attentional control, cognitive inhibition, inhibitory control, working memory, and cognitive flexibility. Higher order executive functions require the simultaneous use of multiple basic executive functions and include planning and fluid intelligence (e.g., reasoning and problem solving), with the total amount of steps to complete multiple tasks being factored into the cognitive risk scores by the correlation model. Further feature descriptions include word finding ability and history, which are also tested via the CAT, and further enable the computing system (through execution of the cognitive correlation model) to generate cognitive risk scores representing each feature description.

Examples described herein achieve a technical effect of utilizing big data and machine learning techniques to more accurately provide individualized cognitive risk assessment for users, particularly for elderly individuals, and determining motor vehicle accident risk on a case by case basis. On a practical level, elderly individuals must typically acquire risk mitigation products at significantly higher rates that steadily increase over time due to an inherently biased assumption that cognitive function generally declines as elderly people grow older. However, for certain elderly individuals, cognitive decline occurs significantly later in life and/or occurs more slowly than others, yet they must still attain risk mitigation products (e.g., car insurance) at higher rates due to such industry generalizations. The techniques described herein can identify these low risk, elderly individuals with exceptional cognitive function through computational analysis of the individual's CAT performance to provide better coverage and/or lower rates for risk mitigation products.

As used herein, a computing device refers to devices corresponding to desktop computers, cellular devices or smartphones, personal digital assistants (PDAs), laptop computers, virtual reality (VR) or augmented reality (AR) headsets, tablet devices, television (IP Television), etc., that can provide network connectivity and processing resources for communicating with the system over a network. A computing device can also correspond to custom hardware, in-vehicle devices, or on-board computers, etc. The computing device can also operate a designated application configured to communicate with the network service.

One or more examples described herein provide that methods, techniques, and actions performed by a computing device are performed programmatically, or as a computer-implemented method. Programmatically, as used herein, means through the use of code or computer-executable instructions. These instructions can be stored in one or more memory resources of the computing device. A programmatically performed step may or may not be automatic.

One or more examples described herein can be implemented using programmatic modules, engines, or components. A programmatic module, engine, or component can include a program, a sub-routine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines.

Some examples described herein can generally require the use of computing devices, including processing and memory resources. For example, one or more examples described herein may be implemented, in whole or in part, on computing devices such as servers, desktop computers, cellular or smartphones, personal digital assistants (e.g., PDAs), laptop computers, VR or AR devices, printers, digital picture frames, network equipment (e.g., routers) and tablet devices. Memory, processing, and network resources may all be used in connection with the establishment, use, or performance of any example described herein (including with the performance of any method or with the implementation of any system).

Furthermore, one or more examples described herein may be implemented through the use of instructions that are executable by one or more processors. These instructions may be carried on a computer-readable medium. Machines shown or described with figures below provide examples of processing resources and computer-readable mediums on which instructions for implementing examples disclosed herein can be carried and/or executed. In particular, the numerous machines shown with examples of the invention include processors and various forms of memory for holding data and instructions. Examples of computer-readable mediums include permanent memory storage devices, such as hard drives on personal computers or servers. Other examples of computer storage mediums include portable storage units, such as CD or DVD units, flash memory (such as carried on smartphones, multifunctional devices or tablets), and magnetic memory. Computers, terminals, network enabled devices (e.g., mobile devices, such as cell phones) are all examples of machines and devices that utilize processors, memory, and instructions stored on computer-readable mediums. Additionally, examples may be implemented in the form of computer-programs, or a computer usable carrier medium capable of carrying such a program.

System Description

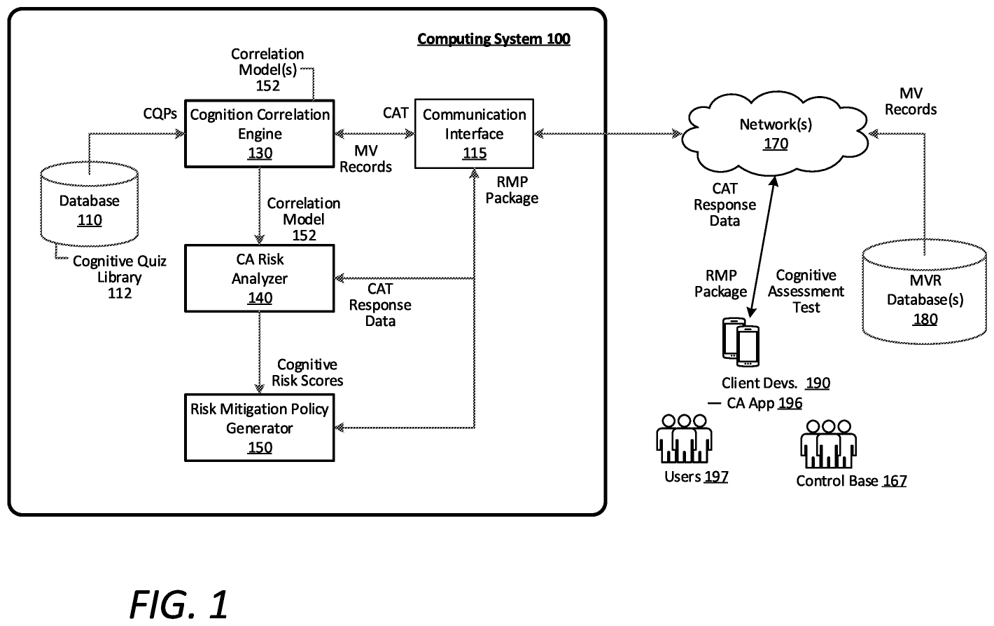

FIG. 1 illustrates a network-based computing system executing a cognition correlation model to generate a cognitive assessment test for determining individualized vehicle accident risk for consumers, according to examples described herein. Referring to FIG. 1, the computing system 100 can include a communication interface 115 that communicates, over one or more networks 170, with client computing devices 190 of control base individuals 167 and inquiring users 197 of a cognitive assessment and risk mitigation service provided by the computing system 100. In various implementations, the control base individuals 197 can comprise volunteers that have agreed to take a cognitive assessment test (CAT) with cognitive quiz problems (CQPs) or questions that test various cognitive functions and abilities of the control base individuals 167. In addition to taking the CAT, the control base individuals 197 also agree to provide the computing system 100 with their motor vehicle records (MVRs) over a past period of time (e.g., three years). In certain examples, the MVRs can comprise official accident data indicating recorded accidents by the control base individuals 167 stored in official databases 180 (e.g., DMV databases or police records). In further examples, the MVR information can include various additional data (e.g., on-board video data) indicative of driving habits, driving abilities, close calls, minor accidents and incidences, and the like.

In various examples, the control base individuals 167 can access the CAT on their client devices 190 via a website or through execution of a cognitive assessment application 196 that provides a user interface presenting each CQP of the CAT. The computing system 100 can include a cognition correlation engine 130 that operates to provide the CAT to the control base individuals 167 as a sequence of CQPs from a cognitive quiz library 112 stored in a database 110 of the computing system 100. The cognition correlation engine 130 can process the MVR information and CAT responses from the control base individuals 167 by executing one or more correlation models 152 that can be trained and tested for accuracy.

The control base individuals 167 can comprise statistically significant representation of the total target populace (e.g., of seniors over the age of sixty-five years old) with a confidence interval of at least 95%. Furthermore, the number of control base individuals 167 can increase over time as more and more people volunteer, or when inquiring users 197 that take the CAT provide the requisite consent for the system 100 to review their MVR information (e.g., over the previous two or three years). In various implementations, the cognition correlation engine 130 can execute the correlation model(s) 152 to discover correlations between the various CAT responses (e.g., correct or incorrect responses) and the MVR information on a collective basis. For example, when a specific CAT response (or aspect of the CAT response, such as taking more than a minute to provide the CAT response) to a particular CQP from several control base individuals 167 that have experienced a traffic accident over the previous year, the cognition correlation engine 130 can identify that CQP as being highly correlative of accident risk for future inquiring users 197. Accordingly, the cognition correlation engine 130 can train, test, and refine the correlation model 152 over time or continuously to become increasingly accurate and more and more predictive of vehicular accident risk for future CAT takers.

As described herein, the CAT can test various cognitive metrics of the control base individuals 167, such as spatial reasoning, motor function, word-finding and language processing ability, and the time it takes to complete each question/assertion as well as the overcall CAT. As such, CAT performance of the individuals in the control base 167 can enable the cognitive correlation engine 130 to generally measure the cognitive well-being of the control base individuals 167 and identify any correlations between such cognitive metrics and vehicular accident risk. The cognition correlation engine 130 can fine tune the correlation model 152 either autonomously (e.g., through machine learning techniques) and/or through manual adjustments by a technical analyst (e.g., via software updates). The result can comprise a highly accurate correlation model 152 executable on CAT response data from any individual (e.g., future inquiring users 197), and that can predict vehicular accident risk simply by the inquiring user 197 taking the CAT and providing basic information (e.g., age and gender information).

According to examples described herein, the computing system 100 can utilize the tuned correlation model 152 to provide a cognitive assessment and risk mitigation service for inquiring users 197. In certain aspects, this service can comprise an insurance service in which risk mitigation packages (e.g., vehicle insurance coverage policies) can be generated and priced in a highly tailored manner based on cognitive risk that is correlated to vehicular accident risk. In doing so, the inquiring user 197 can access the CAT from a client computing device 190, either via a user interface on a website or through a user interface generated through execution of the cognitive assessment application 196. In some aspects, the cognitive quiz questions and/or problems of the CAT can be provided to the inquiring user 197 in a sequential manner (e.g., as true false questions, problem solving tasks, or game-type puzzles).

The inquiring user 197 can provide CAT response data corresponding to answers, puzzle solving, and/or performed tasks in response to each CQP of the CAT. The CAT response data can also indicate the time taken to provide a response to each CQP, whether the inquiring user 197 answered or responded correctly or incorrectly, and/or a degree to which the user 197 responded correctly or incorrectly. In various implementations, the computing system 100 can include a cognitive assessment analyzer 140 that receives the CAT response data from each inquiring user 197 and executes the correlation model 152 on the CAT response data to output a set of cognitive risk scores for the inquiring user 197.

In certain examples, the set of cognitive risk scores can correspond directly to an underwriting class of the inquiring user 197 for vehicular risk mitigation policies. Accordingly, instead of a tedious underwriting process in which the inquiring user 197 must be physically interviewed and motor vehicle records analyzed, the inquiring user 197 may take the CAT. This tedious underwriting process inherently involves a technical problem in that previous underwriting techniques have been unable to classify a customer's risk remotely, without the customer's physical presence and/or vehicle accident history. Thus, the computing system 100 provides a technical solution this this technical problem by utilizing a highly accurate correlation model 152 to analyze CAT response data from users 197 in order to determine vehicular accident risk.

In analyzing the CAT response data through execution of the correlation model 152, the cognitive assessment analyzer 140 can measure the health of the inquiring user's 197 cognitive metrics, such as how well the inquiring user 197 performs with spatial reasoning, motor function, word-finding and language processing ability, as well as how quickly the inquiring user 197 can complete each CQP and the overcall CAT. The cognitive assessment analyzer 140 may then output the cognitive risk scores based on the health of the inquiring user's 197 cognitive metrics to a risk mitigation policy generator 150 of the computing system 100.

Based on the set of cognitive risk scores, the risk mitigation policy generator 150 can determine an underwriting class of the inquiring user 197 (e.g., elite, preferred, standard plus, standard, substandard, high risk, etc.), or an individually tailor a premium and/or risk mitigation policy for the inquiring user 197. For underwriting class implementations, the risk mitigation policy generator 150 can classify the inquiring user 197 based on the cognitive risk scores and generator a risk mitigation policy (RMP) package for the inquiring user 197. This package can comprise different preconfigured vehicle insurance coverage policies with premiums based on the set cognitive risk scores of the inquiring user 197. Thus, if the inquiring user 197 performed well on the CAT, generally the premiums for each risk mitigation policy will be lower. For elderly individuals that have healthy cognitive function, this can provide significant cost savings for automobile risk mitigation.

Additionally or alternatively, the risk mitigation policy generator 150 can provide a customized risk mitigation policy package for the inquiring user 197, which can include added coverage areas, individualized premium pricing, and the like—all based on the cognitive risk scores of the inquiring user 197. In either case, the risk mitigation policy package may be presented to the inquiring user 197 on an interactive customer service interface of the website or cognitive assessment application 196 executing on the client computing device of the inquiring user 197. This interactive customer service interface can include each offered risk mitigation policy in the RMP package, which the inquiring user 197 can view, compare with other RMPs, select a desired RMP, and purchase the selected RMP. Thus, the computing system 100 can provide a single user interface for taking the CAT, generating cognitive risk scores for inquiring user 197, and providing the individualized RMP packages for the inquiring users 197 for review, comparison, and/or purchase.

Client Computing Device

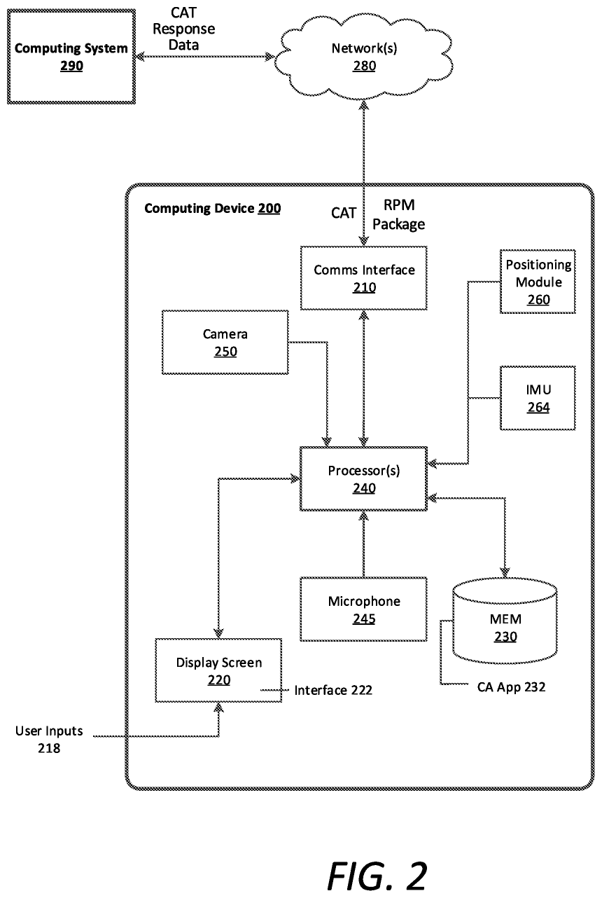

FIG. 2 is a block diagram illustrating an example computing device executing a service application for communicating with a computing system 290 (e.g., the computing system 100 of FIG. 1), according to examples described herein. In many implementations, the computing device 200 can comprise a mobile computing device, such as a smartphone, tablet computer, laptop computer, VR or AR headset device, and the like. As such, the computing device 200 can include telephony features such as a microphone 245, a camera 250, and a communication interface 210 to communicate with external entities using any number of wireless communication protocols. The computing device 200 can further include a positioning module 260 (e.g., GPS) and an inertial measurement unit 264 that includes one or more accelerometers, gyroscopes, or magnetometers. In certain aspects, the computing device 200 can store a designated cognitive assessment application 232 in a memory 230. In variations, the memory 230 can store additional applications executable by one or more processors 240 of the computing device 200, enabling access and interaction with one or more host servers over one or more networks 280. In still further examples, the computing device 200 can provide access to the Internet such that an inquiring user 197 can access a website of the cognitive assessment and risk mitigation service provided by the computing system 290.

Additionally, the computing device 200 can be operated by a control base user 167 or an inquiring user 197 through execution of the cognitive assessment application 232. In various examples, the user 167, 197 can select the cognitive assessment application 232 via a user input 218 on the display screen 220, which can cause the application 232 to be executed by the processor 240. In response, a user application interface 222 can be generated on the display screen 220, which can display the various features of the cognitive assessment and risk mitigation service provided by the computing system 290. One such feature can be selected to present the CAT on the user interface 222. The CAT can comprise a sequential set of cognitive quiz problems (CQPs), which the user 167, 197 can solve or answer.

As provided herein, the application 232 can enable a communication link over one or more networks 280 with the computing system 290, such as the computing system 100 as shown and described with respect to FIG. 1. The processor 240 can generate user interface features 222 using content data received from the computing system 290 over network 280. Furthermore, as discussed herein, the application 232 can enable the computing system 290 to cause the generated user interface 222 to be displayed on the display screen 220.

In various examples, CAT response data corresponding to the user 167, 197 interacting with the CAT can be sequentially transmitted to the computing system 290 over the network 280. For control base users 167, the CAT response data can provide a correlation model executable by the computing system 290 with cognitive data that can be measured against actual MVR information of the control base individuals 167. Thus, in a training and refinement phase, the computing system 290 can tune the correlation model to be highly accurate and predictive of motor vehicle risk based on CAT response data from inquiring users 197.

Upon tuning the correlation model, the computing system 290 can provide the CAT to inquiring users 197. The CAT response data from the inquiring users 197 can be processed to determine a set of cognitive risk scores for each inquiring user 197 and generate an individualized risk mitigation policy (RMP) package for the inquiring user 197. As described herein, the RMP package can include a set of customized risk mitigation policies for the inquiring user 197, or a set of preconfigured risk mitigation policies that have individualized premiums based on the user's 197 cognitive risk scores.

### Methodology

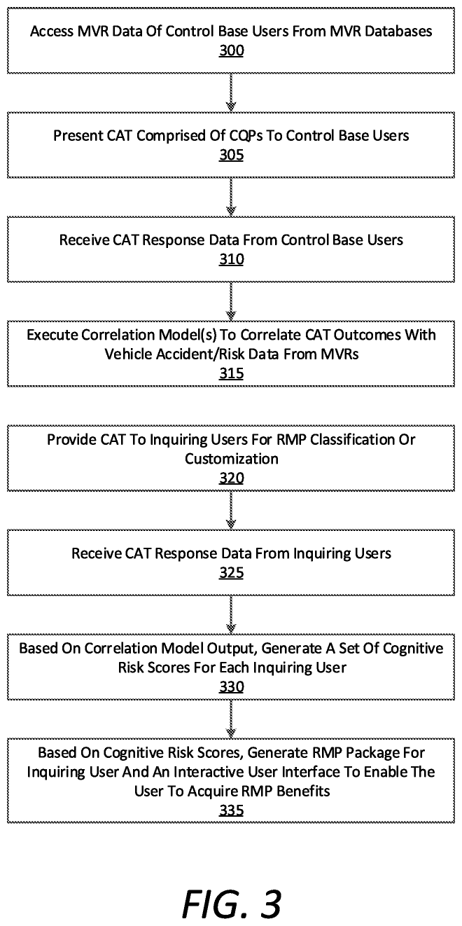

FIG. 3 is a flow chart describing an example method for generating cognitive assessment test through execution of a cognition correlation model and determining individual vehicle accident risk, according to example described herein. In the below discussion of FIG. 3, reference may be made to reference characters representing like features as shown and described with respect to FIGS. 1 and 2. Furthermore, the processes described below in connection with the flow chart of FIG. 3 may be performed by an example computing system 100, 290 as shown and described with respect to FIGS. 1 and 2. Referring to FIG. 3, the computing system 100 can access motor vehicle record (MVR) data of control base individuals 167 who have agreed to take a cognitive assessment test (CAT) from one or more MVR databases 180 (300). In various examples, the computing system 100 can present the CAT, comprised of a number of cognitive quiz problems (CQPs), to the control base users 167 (e.g., via a user interface of a dedicated app or website) (305). For example, the CAT may be presented sequentially (e.g., similar to an IQ test).

The computing system 100 may then receive CAT response data corresponding to the control base users 167 interaction with the CAT (310). The CAT response data can comprise answers to CAT questions (e.g., correct or incorrect multiple-choice selections), performance information (e.g., the manner in which a user 167 solved a particular puzzle or problem and whether the user 167 succeeded), and temporal information (e.g., the time required for the user 167 to solve a particular problem or provide an answer to a particular question). In various examples, the computing system 100 can execute a correlation model 152 to correlate the CAT response data with the MVR information from the control base users 167 (315). For example, execution of the correlation model 152 can enable the computing system 100 to identify correlations between certain cognitive anomalies or issues (e.g., declining motor function or spatial reasoning skills as determined from the CAT response data) and vehicular accident risk.

In various examples, the computing system 100 and/or software developers fine-tuning the correlation model 152 can implement a training and testing phase in which the correlation model 152 gets more and more refined and accurate in predicting vehicular accident risk based on CAT response data alone. When the correlation model 152 has been tested to have reliable accuracy beyond a particular threshold (e.g., 95%) using the MVR information and CAT response data from the control base users 167, the computing system 100 may provide access to the CAT to any inquiring user 197 seeking a risk mitigation policy (e.g., vehicle insurance). Thus, the computing system 100 can provide the CAT to inquiring users 197—who have not provided access to their personal MVR information—for risk mitigation policy classification and/or customization (320).

For each CAT session of each inquiring user 197, the computing system 100 can receive CAT response data corresponding to the user's 197 performance in taking the CAT (325). As described herein, the CAT response data can indicate correct or incorrect selections, problem or puzzle solving performance, and temporal information—all of which assess the spatial reasoning, motor function, word finding and language ability, processing ability, and any other cognitive metric described throughout the present disclosure. The computing system 100 may run the CAT response data for each inquiring user 197 through the correlation model 152, which can output a set of cognitive risk scores for each inquiring user (330). The cognitive risk scores can correspond directly to vehicular accident risk, and can be compiled in a risk table for the inquiring user 197, or the user 197 may be classified in a vehicle accident risk table based on the set of cognitive risk scores. Additionally or alternatively, the output of the correlation model 152 can include an underwriting class of the user 197 based on the CAT response data from the user 197 taking the CAT. Thus, based on the set of cognitive risk scores, the computing system 100 can generate a risk mitigation policy (RMP) package for the inquiring user 197 and an interactive user interface to enable the inquiring user 197 to view, compare, and/or select an RMP (e.g., and any potential benefits, such as discounts, additional coverage, etc.) for purchase (335).

As described herein, the RMP package can comprise various policies offered to the user 197, which can be priced based on the vehicular accident risk of the user 197 as determined by the correlation model 152. In certain examples, the cognitive risk scores can be used by the computing system 100 to determine a risk classification or the user 197, which tier in which the user 197 is to be classified, and/or to generate an individualized set of RMPs for the user 197 with custom prices based on the user's 197 performance in taking the CAT. In various examples, the user 197 may select a desired RMP from the offered package, and elect to purchase the select RMP via the interactive user interface. Accordingly, the interactive user interface can also be custom-generated based on the individualized RMPs created for the user 197—with customized policy details presented, such as coverage areas, coverage breadth, individualized pricing, and the like.

### HARDWARE DIAGRAM

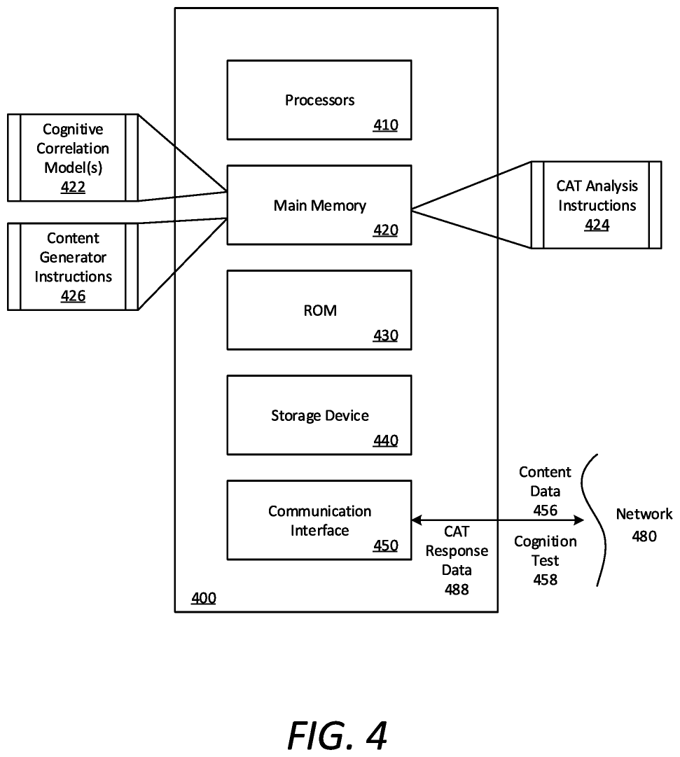

FIG. 4 is a block diagram that illustrates a computer system 400 upon which examples described herein may be implemented. A computer system 400 can be implemented on, for example, a server or combination of servers. For example, the computer system 400 may be implemented as part of a network-based service, such as described in FIGS. 1 through 3. In the context of FIG. 1, the computer system 100 may be implemented using a computer system 400 such as described by FIG. 4. The computer system 100 may also be implemented using a combination of multiple computer systems as described in connection with FIG. 4.

In one implementation, the computer system 400 includes processing resources 410, a main memory 420, a read-only memory (ROM) 430, a storage device 440, and a communication interface 450. The computer system 400 includes at least one processor 410 for processing information stored in the main memory 420, such as provided by a random-access memory (RAM) or other dynamic storage device, for storing information and instructions which are executable by the processor 410. The main memory 420 also may be used for storing temporary variables or other intermediate information during execution of instructions to be executed by the processor 410. The computer system 400 may also include the ROM 430 or other static storage device for storing static information and instructions for the processor 410. A storage device 440, such as a magnetic disk or optical disk, is provided for storing information and instructions.

The communication interface 450 enables the computer system 400 to communicate with one or more networks 480 (e.g., cellular network) through use of the network link (wireless or wired). Using the network link, the computer system 400 can communicate with one or more computing devices, one or more servers, and/or one or more databases. In accordance with examples provided herein, the executable instructions stored in the memory 420 can include cognitive correlation instructions 422, CAT response analysis instructions 424, and content generator instructions 426.

By way of example, the instructions and data stored in the memory 520 can be executed by the processor 410 to implement the functions of an example computing system 100 of FIG. 1. In various examples, the processors 410 can execute the cognitive correlation instructions 422 to process CAT response data 488 from control base users 167 and MVR data from the same users 167 to determine a set of correlations between the cognitive health of the users 167 and vehicular accident risk. The processors 410 can further execute the CAT response analysis instructions 424 to provide the CAT 458 to the inquiring users 197, receive CAT response data 488 from each inquiring user 197, and determine a set of cognitive risk scores that directly correlate to vehicle accident risk. In still further examples, the processors 410 can execute the content generator instructions 426 to generate a RMP package based on the vehicle accident risk of the inquiring user 197 based on the cognitive risk determined from the user's 197 performance on the CAT 458, and then generate a set of user interface features for an interactive user interface to present the RMP package to the user 197 for review, comparison, selection, and/or purchase.

Examples described herein are related to the use of the computer system 400 for implementing the techniques described herein. According to one example, those techniques are performed by the computer system 400 in response to the processor 410 executing one or more sequences of one or more instructions contained in the main memory 420. Such instructions may be read into the main memory 420 from another machine-readable medium, such as the storage device 440. Execution of the sequences of instructions contained in the main memory 420 causes the processor 410 to perform the process steps described herein. In alternative implementations, hard-wired circuitry may be used in place of or in combination with software instructions to implement examples described herein. Thus, the examples described are not limited to any specific combination of hardware circuitry and software.

It is contemplated for examples described herein to extend to individual elements and concepts described herein, independently of other concepts, ideas or systems, as well as for examples to include combinations of elements recited anywhere in this application. Although examples are described in detail herein with reference to the accompanying drawings, it is to be understood that the concepts are not limited to those precise examples. As such, many modifications and variations will be apparent to practitioners skilled in this art. Accordingly, it is intended that the scope of the concepts be defined by the following claims and their equivalents. Furthermore, it is contemplated that a particular feature described either individually or as part of an example can be combined with other individually described features, or parts of other examples, even if the other features and examples make no mentioned of the particular feature. Thus, the absence of describing combinations should not preclude claiming rights to such combinations.

What is claimed is:

1. A computing system implementing a cognitive-based risk assessment service, comprising:
a network communication interface to communicate, over one or more networks, with computing devices of users of the cognitive-based risk assessment service;
one or more processors; and
a memory resource storing instructions that, when executed by the one or more processors, cause the computing system to:
access, over the one or more networks, motor vehicle records of a plurality of control base users;
provide, over the one or more networks, a cognitive assessment test to computing devices of the plurality of control base users;
receive, over the one or more networks, first response data from the computing devices of the plurality of control base users, the first response data corresponding to the plurality of the control base users providing responses to each cognitive quiz problem of the cognitive assessment test;
execute a correlation model to (i) based on the first response data from each control base user, generate a set of cognitive risk scores for the control base user, the set of cognitive risk scores corresponding to a number of cognitive health metrics of the control base user, and (ii) determine a set of correlations between the cognitive health metrics of the plurality of control base users and vehicle accident risk as indicated in the motor vehicle records of the plurality of control base users;
provide, over the one or more networks, the cognitive assessment test to computing devices of inquiring users of the cognitive-based risk assessment service;
for each inquiring user, receive, over the one or more networks, second response data from the computing device of the inquiring user, the second response data corresponding to the inquiring user providing responses to each cognitive quiz problem in the cognitive assessment test;
using the second response data, execute the correlation model to determine a set of cognitive risk scores corresponding to the cognitive health metrics for the inquiring user, the set of cognitive risk scores further corresponding to motor vehicle risk of the inquiring user based on the cognitive health metrics as determined from the second response data; and
based on the motor vehicle risk of the inquiring user, generate a risk mitigation policy package for the inquiring user on an interactive user interface displayed on the computing device of the inquiring user, the interactive user interface enabling the inquiring user to select a particular risk mitigation policy from the risk mitigation policy package.

2. The computing system of claim 1, wherein the cognitive health metrics comprise at least one of spatial reasoning ability, motor function, word-finding and language processing ability, memory and recall, or reaction time.

3. The computing system of claim 1, wherein each risk mitigation policy in the risk mitigation policy package for the inquiring user is customized for the inquiring user based on the motor vehicle risk of the inquiring user as determined from the second response data.

4. The computing system of claim 1, wherein risk mitigation policy in the risk mitigation policy package for the inquiring user is individually priced based on the motor vehicle risk of the inquiring user as determined from the second response data.

5. The computing system of claim 1, wherein the risk mitigation policy package is generated for the inquiring user based on an underwriting class of the user as determined from the motor vehicle risk of the inquiring user, which is determined from the second response data.

6. The computing system of claim 1, wherein execution of the correlation model using the first response data causes the computing system to test an accuracy of the correlation model against the motor vehicle records of the plurality of control base users.

7. A non-transitory computer readable medium storing instructions that, when executed by one or more processors, cause the one or more processors to:
communicate, over one or more networks, with computing devices of users of a cognitive-based risk assessment service;
access, over the one or more networks, motor vehicle records of a plurality of control base users;
provide, over the one or more networks, a cognitive assessment test to computing devices of the plurality of control base users;
receive, over the one or more networks, first response data from the computing devices of the plurality of control base users, the first response data corresponding to the plurality of the control base users providing responses to each cognitive quiz problem of the cognitive assessment test;
execute a correlation model to (i) based on the first response data from each control base user, generate a set of cognitive risk scores for the control base user, the set of cognitive risk scores corresponding to a number of cognitive health metrics of the control base user, and (ii) determine a set of correlations between the cognitive health metrics of the plurality of control base users and vehicle accident risk as indicated in the motor vehicle records of the plurality of control base users;
provide, over the one or more networks, the cognitive assessment test to computing devices of inquiring users of the cognitive-based risk assessment service;
for each inquiring user, receive, over the one or more networks, second response data from the computing device of the inquiring user, the second response data corresponding to the inquiring user providing responses to each cognitive quiz problem in the cognitive assessment test;
using the second response data, execute the correlation model to determine a set of cognitive risk scores corresponding to the cognitive health metrics for the inquiring user, the set of cognitive risk scores further corresponding to motor vehicle risk of the inquiring user based on the cognitive health metrics as determined from the second response data; and
based on the motor vehicle risk of the inquiring user, generate a risk mitigation policy package for the inquiring user on an interactive user interface displayed on the computing device of the inquiring user, the interactive user interface enabling the inquiring user to select a particular risk mitigation policy from the risk mitigation policy package.

8. The non-transitory computer readable medium of claim 7, wherein the cognitive health metrics comprise at least one of spatial reasoning ability, motor function, word-finding and language processing ability, memory and recall, or reaction time.

9. The non-transitory computer readable medium of claim 7, wherein each risk mitigation policy in the risk mitigation policy package for the inquiring user is customized for the inquiring user based on the motor vehicle risk of the inquiring user as determined from the second response data.

10. The non-transitory computer readable medium of claim 7, wherein risk mitigation policy in the risk mitigation policy package for the inquiring user is individually priced based on the motor vehicle risk of the inquiring user as determined from the second response data.

11. The non-transitory computer readable medium of claim 7, wherein the risk mitigation policy package is generated for the inquiring user based on an underwriting class of the user as determined from the motor vehicle risk of the inquiring user, which is determined from the second response data.

12. The non-transitory computer readable medium of claim 7, wherein execution of the correlation model using the first response data causes the computing system to test an accuracy of the correlation model against the motor vehicle records of the plurality of control base users.

13. A computer-implemented method of implementing a cognitive-based risk assessment service, the method being perform by one or more processors and comprising:
communicating, over one or more networks, with computing devices of users of a cognitive-based risk assessment service;
accessing, over the one or more networks, motor vehicle records of a plurality of control base users;
providing, over the one or more networks, a cognitive assessment test to computing devices of the plurality of control base users;
receiving, over the one or more networks, first response data from the computing devices of the plurality of control base users, the first response data corresponding to the plurality of the control base users providing responses to each cognitive quiz problem of the cognitive assessment test;
executing a correlation model to (i) based on the first response data from each control base user, generate a set of cognitive risk scores for the control base user, the set of cognitive risk scores corresponding to a number of cognitive health metrics of the control base user, and (ii) determine a set of correlations between the cognitive health metrics of the plurality of control base users and vehicle accident risk as indicated in the motor vehicle records of the plurality of control base users;
providing, over the one or more networks, the cognitive assessment test to computing devices of inquiring users of the cognitive-based risk assessment service;
for each inquiring user, receiving, over the one or more networks, second response data from the computing device of the inquiring user, the second response data corresponding to the inquiring user providing responses to each cognitive quiz problem in the cognitive assessment test;
using the second response data, executing the correlation model to determine a set of cognitive risk scores corresponding to the cognitive health metrics for the inquiring user, the set of cognitive risk scores further corresponding to motor vehicle risk of the inquiring user based on the cognitive health metrics as determined from the second response data; and
based on the motor vehicle risk of the inquiring user, generating a risk mitigation policy package for the inquiring user on an interactive user interface displayed on the computing device of the inquiring user, the interactive user interface enabling the inquiring user to select a particular risk mitigation policy from the risk mitigation policy package.

14. The method of claim 13, wherein the cognitive health metrics comprise at least one of spatial reasoning ability, motor function, word-finding and language processing ability, memory and recall, or reaction time.

15. The method of claim 13, wherein each risk mitigation policy in the risk mitigation policy package for the inquiring user is customized for the inquiring user based on the motor vehicle risk of the inquiring user as determined from the second response data.

16. The method of claim 13, wherein risk mitigation policy in the risk mitigation policy package for the inquiring user is individually priced based on the motor vehicle risk of the inquiring user as determined from the second response data.

17. The method of claim 13, wherein the risk mitigation policy package is generated for the inquiring user based on an underwriting class of the user as determined from the motor vehicle risk of the inquiring user, which is determined from the second response data.

18. The method of claim 13, wherein execution of the correlation model using the first response data causes the computing system to test an accuracy of the correlation model against the motor vehicle records of the plurality of control base users.
